(19) United States
(12) Patent Application Publication
(10) Pub. No.: US 2010/0249715 A1
(43) Pub. Date: Sep. 30, 2010

(54) Device For Assisting With Intravenous Administration

(76) Inventors: Kenneth Langieri, Oak Ridge, NJ (US); Josephine Langieri, Oak Ridge, NJ (US)

Correspondence Address: HODGSON RUSS LLP;THE GUARANTY BUILDING, 140 PEARL STREET, SUITE 100, BUFFALO, NY, 14202-4040, US

(21) Appl. No.: 12/558,917

(22) Filed: Sep. 14, 2009

Related U.S. Application Data
(60) Provisional application No. 61/096,457, filed on Sep. 12, 2008.

(51) Int. Cl. A61M 39/00

(52) U.S. Cl. 604/179

(57) ABSTRACT

A medical tube retaining device has a flexible portion having one or more clips formed thereon for organizing, securing and retaining one or more medical tubes to the flexible portion. An adjustable band is attached to the flexible portion for securing the device to a limb of a patient.

### FIELD OF THE INVENTION

The present invention relates generally to the medical field and more particularly to intravenous (IV) tubes and their use. Specifically, the present invention relates to a device for organizing and securing one or more IV tubes.

### BACKGROUND OF THE INVENTION

It is common medical practice to treat and medicate a patient by introducing therapeutic agents, drugs, medications, nutrients, and various other liquids directly into the blood stream of the patient through systems commonly known as intravenous (IV) administration systems. Often a critically ill patient will require several such systems concurrently operative to provide different treatment agents. One problem which may arise from a patient having several IV tubes is the interaction, or entanglement, of the tubes which may result in one or more of the tubes becoming disconnected from the patient or the IV reservoir. Entanglement of the tubes may be caused by a patient moving while in a conscious or unconscious state. Normally, the IV tubes are taped to the patient with adhesive tape in an attempt to hold them firmly in position and reduce pain to the patient from movement of the tubes. However, the adhesive tape constantly pulls on the patient's skin which is uncomfortable even in the best of circumstances. Movement of the tubes may result in injury to the patient or failure to infuse the patient with the required medications.

It is therefore desirable to have a medical tube retaining device to which one or more IV tubes may be secured while keeping the tubes separate and unentangled from each other. The device should also be easily fixed to the patient's limb, such as an arm or leg. The device should also be easily transportable with the IV reservoirs as the patient moves from location to location. The device should also be simple in construction, easy to apply, and economical to manufacture.

### BRIEF SUMMARY OF THE INVENTION

The medical tube retaining device may include an adjustable band adapted to be secured to a patient's body near an intravenous therapy location. The band may be connected to a flexible portion, or tube holder, having one or more clips for securing and retaining one or more IV tubes to the holder.

### BRIEF DESCRIPTION OF THE SEVERAL VIEWS OF THE DRAWINGS

FIG. 1 is a perspective view of the medical tube retaining device of the present invention.

FIG. 2A is a side view of the flexible portion of the medical tube retaining device of the present invention.

FIG. 2B is a close-up view of a clip attached to the flexible portion of FIG. 2A.

### DETAILED DESCRIPTION OF THE INVENTION

The medical tube retaining device 10 may have a band 20 sized to encircle the limb or other body part of a patient. The band 20 is preferably constructed of a hook and loop fastener material, but may also be constructed of an elastic woven, knit, or webbing material, or other suitable material. The band 20 should be adjustable to fit around a patient's limb to which the device will be secured. The band should be easily adjustable by the patient or a nurse, doctor, or other medical staff attending to the patient.

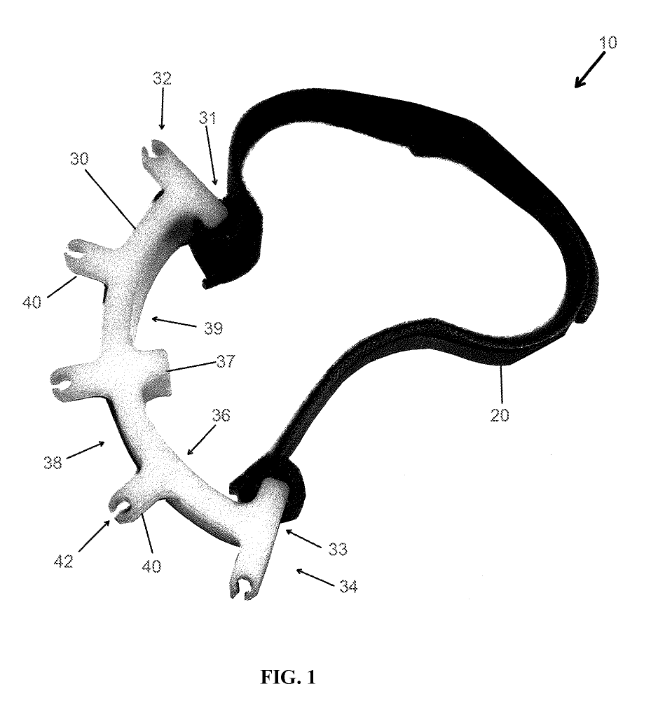

As shown in FIG. 1, the band 20 may be attached to a flexible portion, or tube holder, 30. Preferably, the band 20 is attached at opposing ends 32, 24 of the flexible portion. The band 20 may be secured through apertures 31, 33 formed in the opposing ends 32, 34 of the flexible portion 30.

The flexible portion 30 may be constructed of a polyvinyl plastic or other suitable material, and may be flexible in order to fit around the arm or other limb of the patient. The flexible portion 30 may be generally curved in shape to conform to the patient's limb. The concave surface 36 of the flexible portion 30 may be adapted to rest along the limb of the patient. One or more protrusions 37 may be formed on the concave surface 36. The protrusions 37 may contact the patient's limb allowing airflow through the spaces 39 formed between the protrusions 37.

The convex surface 38 of the flexible portion 30 may have one or more clips 40 for holding the IV tubes (not shown.) The clips 40 have openings 42 into which the IV tubes may be easily inserted or removed. The clips 40 provide sufficient frictional clasping of the IV tube to prevent the tube from falling away from the device 10, but not so much friction that the tubing is not readily released from the clips 40. The clips 40 may be formed integrally with the flexible portion 30 and constructed of the same flexible material.

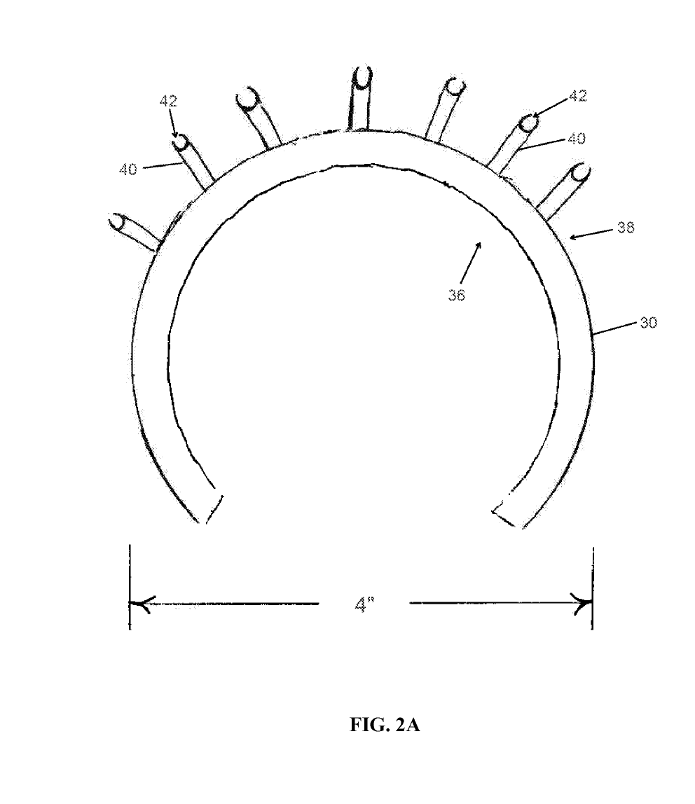

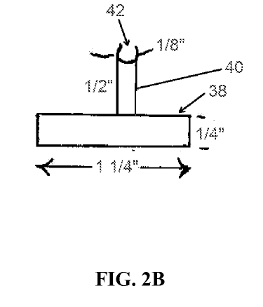

An exemplary model of the flexible portion 30 is shown in FIGS. 2A and 2B. In this example, the flexible portion 30 may be formed with seven clips 40. The overall width of the flexible portion 30 may be approximately 4″ if the device is meant to be attached to a patient's arm. However, it should be understood that the device may be constructed in any size needed in order to accommodate different size patients and different body parts to which the device may be secured.

A single clip 40, attached to a section of the flexible portion 30, is shown in FIG. 2B. In this example, the flexible portion 30 is approximately ¼″ thick. The clips 40 may protrude approximately ½″ from the convex surface 38 of the flexible portion 30, and the clips 40 may be approximately ⅛″ in diameter. The clips 40 may also be spaced approximately 1¼″ apart from each other. This is to ensure organization and unentaglement of the IV tubes.

Although the present invention has been described with respect to one or more particular embodiments, it will be understood that other embodiments of the present invention can be made without departing from the spirit and scope of the present invention.

What is claimed is:

1. A medical tube retaining device comprising:
a flexible portion having one or more clips formed thereon for organizing, securing and retaining one or more medical tubes to the flexible portion; and
an adjustable band attached to the flexible portion for securing the device to a limb of a patient.

2. The medical tube retaining device of claim 1, wherein the flexible portion has a generally curved shape defining a concave surface and a convex surface.

3. The medical tube retaining device of claim 2, wherein the concave surface has protrusions formed thereon.

4. The medical tube retaining device of claim 1 wherein the clips have openings for receiving the medical tube.

5. The medical tube retaining device of claim 1 wherein the flexible portion is constructed from polyvinyl plastic.

6. The medical tube retaining device of claim 1 wherein the adjustable band includes a hook and loop fastener portion for attachment and detachment to and from the patient's limb.
